(19) United States
(12) Patent Application Publication
(10) Pub. No.: US 2026/0144780 A1
(43) Pub. Date: May 28, 2026

(54) COMPOSITION FOR ENHANCING ADRENOMEDULLIN GENE EXPRESSION

(71) Applicant: HOUSE WELLNESS FOODS CORPORATION, Itami-shi (JP)

(72) Inventors: Haruka NAKANISHI, Itami-shi (JP); Kazuki ASADA, Itami-shi (JP); Tatsuya OHARA, Itami-shi (JP); Koutarou MUROYAMA, Itami-shi (JP)

(73) Assignee: HOUSE WELLNESS FOODS CORPORATION, Itami-shi (JP)

(21) Appl. No.: 19/406,509

(22) Filed: Dec. 2, 2025

Related U.S. Application Data
(62) Division of application No. 18/037,107.

(30) Foreign Application Priority Data
Nov. 16, 2020 (JP) 2020-189986

(51) Int. Cl. A61K 31/4525; A61K 31/10; A61K 31/11; A61K 31/12; A61K 31/165; A61K 31/202; A61K 31/26; A61K 31/404; A61P 43/00

(52) U.S. Cl. CPC A61K 31/4525; A61K 31/10; A61K 31/11; A61K 31/12; A61K 31/165; A61K 31/202; A61K 31/26; A61K 31/404; A61P 43/00

(57) ABSTRACT

The present invention provides a composition having an action to enhance adrenomedullin gene expression. One or more embodiments of the present invention relate to a composition for enhancing adrenomedullin gene expression containing a TRPV1 agonist and a TRPA1 agonist as active ingredients. The TRPV1 agonist can be, for example, at least one selected from capsaicin, 6-shogaol, 6-gingerol and piperine. The TRPA1 agonist can be, for example, at least one selected from allyl isothiocyanate, cinnamaldehyde, diallyl disulfide, ASP7663 and oxylipin.

### SEQUENCE LISTING

The instant application contains a Sequence Listing which has been submitted in XML format via Patent Center and is hereby incorporated by reference in its entirety. Said XML copy is named 125315-5017-DIV.xml, created on Dec. 2, 2025 and is 9,260 bytes.

### TECHNICAL FIELD

The present invention relates to a composition for enhancing adrenomedullin gene expression.

### BACKGROUND ART

Adrenomedullin is a vascular regulatory peptide having vasodilator activity. Adrenomedullin is produced in various organs of, e.g., the circulatory system and digestive system, and reported to have important physiological activities such as vasodilation, angiogenesis, antimicrobial action, anti-bowel inflammation, gastric mucosa protection and suppression of thrombogenesis (Patent Literature 1).

TRP (transient receptor potential) channels are ion channels (molecule) passing cations such as sodium ions and calcium ions. TRP channels are activated by, e.g., a temperature change, a mechanical stimulation or an oxidative stress. Many homologs of TRP channels have been identified and a TRP channel superfamily includes 6 subfamilies. TRPV1 belongs to the TRPV subfamily and TRPA1 belongs to the TRPA subfamily.

Patent Literature 1 discloses an adrenomedullin production enhancer containing a compound derived from ginseng, a compound derived from dried ginger and/or a compound derived from Sansho (Japanese pepper) as an active ingredient.

Non Patent Literature 1 discloses that a kind of herbal medicine Daiken chuto (containing Sansho, dried ginger and ginseng) promotes release of adrenomedullin from the intestinal epithelial cells, thereby promoting blood flow in the intestine; and that Daiken chuto and a component thereof, 6-shogaol, have an action to promote release of adrenomedullin from the intestinal epithelial cells and the action is inhibited by a TRPA1 antagonist.

Patent Literature 2 discloses an adipocyte differentiation inducer and a functional food for preventing or improving (treating) diseases caused by abnormal differentiation of adipocytes, containing an agonist to a TRP calcium channel protein as an active ingredient. The Literature discloses TRPV1, TRPV4, TRPM8 and TRPA1 as examples of the TRP calcium channel protein, and capsaicin, menthol, allyl isothiocyanate, cinnamaldehyde and alliin as examples of the agonist.

Patent Literature 3 discloses a method for improving taste of an oral care composition, comprising mixing a TRPV1 activator and a bad taste agent such as a metal salt. The Literature discloses capsaicin, shogaol, gingerol and piperine as examples of the TRPV1 activator.

Patent Literature 4 discloses a composition containing a TRPV1 channel activator, a TRPA1 channel activator, an ASIC channel activator, or a combination thereof, for treating, e.g., pathological conditions of the peripheral/central nervous systems and painful muscle contraction. The Literature discloses capsaicin and gingerol as examples of the TRPV1 channel activator, and allyl isothiocyanate, cinnamaldehyde, diallyl sulfide and sanshool as examples of the TRPA1 channel activator.

Patent Literature 5 discloses a TRPA1 stimulant containing a fatty acid having 12 to 26 carbon atoms, at least one carbon-carbon double bond and at least one functional group selected from the group consisting of a hydroxyl group and a hydroperoxide group, as an active ingredient. Patent Literature 5 discloses that the TRPA1 stimulant can be added to a food as a food additive to alter/improve taste. Patent Literature 5 discloses that the fatty acid can be extracted from a plant as an oxylipin; that the fatty acid is, for example, at least one selected from the group consisting of (10E,12Z)-9-hydroxy-10,12-octadecadienoic acid, (9Z,11E)-13-hydroxy-9,11-octadecadienoic acid, (9Z,11E,15Z)-13-hydroxy-9,11,15-octadecatrienoic acid, (10E,12Z)-9-hydroperoxy-10,12-octadecadienoic acid, (9Z,11E)-13-hydroperoxy-9,11-octadecadienoic acid, (9Z,11E,15Z)-13-hydroperoxy-9,11,15-octadecatrienoic acid, (5Z,8Z,11Z,13E)-15-hydroperoxy-5,8,11,13-eicosatetraenoic acid, (4Z,7Z,10Z,13Z,15E,19Z)-17-hydroperoxy-4,7,10,13,15,19-docosahexaenoic acid, (9Z)-12-hydroxy-9-octadecenoic acid and (9E)-12-hydroxy-9-octadecenoic acid.

### CITATION LIST

### Patent Literature

Patent Literature 1: International Publication WO2009/104248
Patent Literature 2: JP Patent Publication No. 2006-199647A
Patent Literature 3: JP Patent Publication No. 2012-524792A
Patent Literature 4: JP Patent Publication No. 2017-513864A
Patent Literature 5: JP Patent Publication No. 2014-076979A

### Non Patent Literature

Non Patent Literature 1: Am J Physiol Gastrointest Liver Physiol 304: G428-G436, 2013

### SUMMARY OF INVENTION

### Technical Problem

An object of the present invention is to provide a composition having an action to enhance adrenomedullin gene expression.

### Solution to Problem

The present inventors have found that a composition having a TRPV1 agonist and a TRPA1 agonist as active ingredients has an action to synergetically enhance adrenomedullin gene expression. Based on the finding, the present invention has been accomplished.
(1) A composition for enhancing adrenomedullin gene expression, comprising a TRPV1 agonist and a TRPA1 agonist as active ingredients.
(2) The composition according to (1), wherein the TRPV1 agonist is at least one selected from capsaicin, 6-shogaol, 6-gingerol and piperine.
(3) The composition according to (1) or (2), wherein the TRPA1 agonist is at least one selected from allyl isothiocyanate, cinnamaldehyde, diallyl disulfide, ASP7663 and oxylipin.
(4) Use of a TRPV1 agonist and a TRPA1 agonist for the manufacture of a composition for enhancing adrenomedullin gene expression.
(5) The use according to (4), wherein the composition for enhancing adrenomedullin gene expression is a composition for enhancing adrenomedullin gene expression in cells in vivo or in vitro.
(6) The use according to (5), wherein the cells are epithelial cells such as small intestine epithelial cells.
(7) Use of a TRPV1 agonist and a TRPA1 agonist for the manufacture of a medicament for enhancing adrenomedullin gene expression.
(8) The use according to (7), wherein the medicament for enhancing adrenomedullin gene expression is a medicament for achieving at least one effect selected from increasing blood flow, an antimicrobial action, anti-bowel inflammation, gastric mucosa protection and suppression of thrombogenesis in a subject such as a human or a non-human mammal.
(9) A method for enhancing adrenomedullin gene expression, comprising administering a TRPV1 agonist and a TRPA1 agonist to a subject, such as a human or a non-human mammal, in need of enhancement of or being desirable to enhance adrenomedullin gene expression.
(10) A method for treating or preventing a disease or symptom that is improved by enhancing adrenomedullin gene expression, comprising administering a TRPV1 agonist and a TRPA1 agonist to a subject, such as a human or a non-human mammal, in need of the treatment or prevention of or being desirable to treat or prevent the disease or symptom.
(11) The method according to (10), wherein the disease or symptom is at least one selected from a disease or symptom that is improved by increasing blood flow, a disease or symptom that is caused by a microorganism, bowl inflammation, a disease or symptom that is improved by protecting gastric mucosa, and a disease or symptom that is improved by suppressing thrombogenesis.
(12) A method for enhancing adrenomedullin gene expression in cells in vivo or in vitro, comprising providing a TRPV1 agonist and a TRPA1 agonist to cells in vivo or in vitro.
(13) The method according to (12), wherein the cells are epithelial cells such as small intestine epithelial cells.
(14) A combination of a TRPV1 agonist and a TRPA1 agonist for use in enhancing adrenomedullin gene expression in a subject such as a human and a non-human mammal.
(15) The combination according to (14), being a composition comprising a TRPV1 agonist and a TRPA1 agonist, or a kit comprising a TRPV1 agonist and a TRPA1 agonist not mixed with each other.
(16) A combination of a TRPV1 agonist and a TRPA1 agonist for use in treating or preventing a disease or symptom that is improved by enhancement of adrenomedullin gene expression in a subject such as a human and a non-human mammal.
(17) The combination according to (16), wherein the disease or symptom is at least one selected from a disease or symptom that is improved by increasing blood flow, a disease or symptom that is caused by a microorganism, bowl inflammation, a disease or symptom that is improved by protecting gastric mucosa, and a disease or symptom that is improved by suppressing thrombogenesis.
(18) The combination according to (16) or (17), being a composition comprising a TRPV1 agonist and a TRPA1 agonist, or a kit comprising a TRPV1 agonist and a TRPA1 agonist not mixed with each other.
(19) A combination of a TRPV1 agonist and a TRPA1 agonist for use in enhancing adrenomedullin gene expression in cells in vivo or in vitro.
(20) The combination according to (19), wherein the cells are, epithelial cells such as small intestine epithelial cells.
(21) The combination according to (19) or (20), being a composition comprising a TRPV1 agonist and a TRPA1 agonist, or a kit comprising a TRPV1 agonist and a TRPA1 agonist not mixed with each other.
(22) A pharmaceutical composition, food and beverage composition or cosmetic composition, comprising preferably 0.1 to 95 mass % and more preferably 1 to 50 mass % of a TRPV1 agonist and a TRPA1 agonist based on a total amount of the composition, and at least one additional component which is acceptable as a pharmaceutical, food and drink or a cosmetic.
(23) The pharmaceutical composition, food and beverage composition or cosmetic composition according to (22), wherein the content of the TRPA1 agonist per mole of the TRPV1 agonist is preferably 0.1 to 100 moles, more preferably 0.1 to 50 moles, more preferably 0.1 to 20 moles, more preferably 0.2 to 5 moles and particularly preferably 0.2 to 3 moles.
(24) The pharmaceutical composition, food and beverage composition or cosmetic composition according to (22) or (23), wherein the at least one additional component is at least one selected from a sweetener, an acidulant, vitamins, minerals, a thickener, an emulsifier, an antioxidant, water, a pigment, a flavor, a preservative, an antiseptic agent, a fungicide and other physiological active substances.
(25) A medium composition for cell culture, comprising
at least one medium component,
a TRPV1 agonist and
a TRPA1 agonist.
(26) The medium composition according to (25), wherein the concentration of the TRPV1 agonist is 1 to 100 μM and the concentration of the TRPA1 agonist is 1 to 300 μM.
(27) The medium composition according to (25) or (26), wherein the content of the TRPA1 agonist per mole of the TRPV1 agonist is preferably 0.1 to 100 moles, more preferably 0.1 to 50 moles, more preferably 0.1 to 20 moles, more preferably 0.2 to 5 moles and particularly preferably 0.2 to 3 moles.
(28) Any one of the embodiments of (4) to (27), wherein the TRPV1 agonist is at least one selected from capsaicin, 6-shogaol, 6-gingerol and piperine.
(29) Any one of the embodiments of (4) to (28), wherein the TRPA1 agonist is at least one selected from allyl isothiocyanate, cinnamaldehyde, diallyl disulfide, ASP7663 and oxylipin.

The specification includes the contents disclosed in JP Patent Application No. 2020-189986 based on which the priority of the present application was claimed.

### Advantageous Effects of Invention

According to the present invention, there is provided a composition having an action to enhance adrenomedullin gene expression.

### BRIEF DESCRIPTION OF DRAWINGS

FIG. 1 shows relative adrenomedullin mRNA expression levels to Q actin in rat-derived small intestinal epithelial cell lines treated with 30 μM capsaicin alone, 30 μM or 60 μM AITC alone, or a combination of 30 μM capsaicin and 30 μM or 60 μM AITC (the expression level in a control condition is expressed as 1).

FIG. 2 shows relative adrenomedullin mRNA expression levels to β-actin in rat-derived small intestinal epithelial cell lines treated with 30 μM capsaicin alone, 30 μM or 60 μM cinnamaldehyde alone, or a combination of 30 μM capsaicin and 30 μM or 60 μM cinnamaldehyde (the expression level in a control condition is expressed as 1).

FIG. 3 shows relative adrenomedullin mRNA expression levels to β-actin in rat-derived small intestinal epithelial cell lines treated with 30 μM capsaicin alone, 30 μM or 60 μM diallyl disulfide alone, or a combination of 30 μM capsaicin and 30 μM or 60 μM diallyl disulfide (the expression level in a control condition is expressed as 1).

FIG. 4 shows relative adrenomedullin mRNA expression levels to β-actin in rat-derived small intestinal epithelial cell lines treated with 30 μM capsaicin alone, 10 μM or 20 μM ASP7663 alone, or a combination of 30 μM capsaicin and 10 μM or 20 μM ASP7663 (the expression level in a control condition is expressed as 1).

FIG. 5 shows relative adrenomedullin mRNA expression levels to β-actin in rat-derived small intestinal epithelial cell lines treated with 5 μM 6-shogaol alone, 30 μM or 60 μM AITC alone, or a combination of 5 μM 6-shogaol and 30 μM or 60 μM AITC (the expression level in a control condition is expressed as 1).

FIG. 6 shows relative adrenomedullin mRNA expression levels to β-actin in rat-derived small intestinal epithelial cell lines treated with 5 μM 6-shogaol alone, 30 μM or 60 μM cinnamaldehyde alone, or a combination of 5 μM 6-shogaol and 30 μM or 60 μM cinnamaldehyde (the expression level in a control condition is expressed as 1).

FIG. 7 shows relative adrenomedullin mRNA expression levels to β-actin in rat-derived small intestinal epithelial cell lines treated with 5 μM 6-shogaol alone, 30 μM or 60 μM diallyl disulfide alone, or a combination of 5 μM 6-shogaol and 30 μM or 60 μM diallyl disulfide (the expression level in a control condition is expressed as 1).

FIG. 8 shows relative adrenomedullin mRNA expression levels to β-actin in rat-derived small intestinal epithelial cell lines treated with 5 μM 6-shogaol alone, 10 μM or 20 μM ASP7663 alone, or a combination of 5 μM 6-shogaol and 10 μM or 20 μM ASP7663 (the expression level in a control condition is expressed as 1).

FIG. 9 shows relative adrenomedullin mRNA expression levels to β-actin in rat-derived small intestinal epithelial cell lines treated with 30 μM 6-gingerol alone, 30 μM or 60 μM AITC alone, or a combination of 30 μM 6-gingerol and 30 μM or 60 μM AITC (the expression level in a control condition is expressed as 1).

FIG. 10 shows relative adrenomedullin mRNA expression levels to β-actin in rat-derived small intestinal epithelial cell lines treated with 30 μM 6-gingerol alone, 30 μM or 60 μM cinnamaldehyde alone, or a combination of 30 μM 6-gingerol and 30 μM or 60 μM cinnamaldehyde (the expression level in a control condition is expressed as 1).

FIG. 11 shows relative adrenomedullin mRNA expression levels to β-actin in rat-derived small intestinal epithelial cell lines treated with 30 μM 6-gingerol alone, 60 μM diallyl disulfide alone, or a combination of 30 μM 6-gingerol and 60 μM diallyl disulfide (the expression level in a control condition is expressed as 1).

FIG. 12 shows relative adrenomedullin mRNA expression levels to β-actin in rat-derived small intestinal epithelial cell lines treated with 30 μM 6-gingerol alone, 10 μM or 20 μM ASP7663 alone, or a combination of 30 μM 6-gingerol and 10 μM or 20 μM ASP7663 (the expression level in a control condition is expressed as 1).

FIG. 13 shows relative adrenomedullin mRNA expression levels to β-actin in rat-derived small intestinal epithelial cell lines treated with 30 μM piperine alone, 30 μM or 60 μM AITC alone, or a combination of 30 μM piperine and 30 μM or 60 μM AITC (the expression level in a control condition is expressed as 1).

FIG. 14 shows relative adrenomedullin mRNA expression levels to β-actin in rat-derived small intestinal epithelial cell lines treated with 30 μM piperine alone, 30 μM or 60 μM cinnamaldehyde alone, or a combination of 30 μM piperine and 30 μM or 60 μM cinnamaldehyde (the expression level in a control condition is expressed as 1).

FIG. 15 shows relative adrenomedullin mRNA expression levels to β-actin in rat-derived small intestinal epithelial cell lines treated with 30 μM piperine alone, 30 μM or 60 μM diallyl disulfide alone, or a combination of 30 μM piperine and 30 μM or 60 μM diallyl disulfide (the expression level in a control condition is expressed as 1).

FIG. 16 shows relative adrenomedullin mRNA expression levels to β-actin in rat-derived small intestinal epithelial cell line treated with 30 μM piperine alone, 10 μM or 20 μM ASP7663 alone, or a combination of 30 μM piperine and 10 μM or 20 μM ASP7663 (the expression level in a control condition is expressed as 1).

FIG. 17 shows the results of Experiment 3, Evaluation 1.

FIG. 18 shows the results of Experiment 3, Evaluation 2.

FIG. 19 shows the results of Experiment 3, Evaluation 3.

FIG. 20 shows the results of Experiment 3, Evaluation 4.

FIG. 21 shows the results of Experiment 3, Evaluation 5.

FIG. 22 shows adrenomedullin mRNA expression levels in rat-derived small intestinal epithelial cell lines treated with 5 μM 6-shogaol alone, 20 μM ASP7663 alone, 50 μM, 83.3 μM and 250 μM purified oxylipin alone, or combinations of 5 μM 6-shogaol and 50 μM, 83.3 μM and 250 μM purified oxylipin (the expression level in a control condition is expressed as 1).

### DESCRIPTION OF EMBODIMENTS

### <TRPV1 Agonist>

A TRPV1 agonist is not particularly limited as long as it is a compound having an action to activate TRPV1 (transient receptor potential vanilloid 1). A TRPV1 agonist may be naturally or non-naturally occurring compound.

When a naturally occurring TRPV1 agonist is used, the TRPV1 agonist may be chemically synthesized or derived from a natural raw material, such as a plant and a microorganism, containing the TRPV1 agonist. The form of the TRPV1 agonist derived from a natural raw material is not particularly limited. The TRPV1 agonist may be present in the form of, e.g., a natural raw material, an extract from the natural raw material, or a compound obtained from the natural raw material by purification or concentration.

The TRPV1 agonist can be, for example, a vanilloid, an alkaloid or α,β-unsaturated dialdehyde and preferably at least one selected from a vanilloid and an alkaloid.

The vanilloid may be preferably at least one selected from capsaicinoid, 6-shogaol, 6-gingerol, 6-paradol, zingerone and derivatives thereof and particularly preferably at least one selected from capsaicinoid, 6-shogaol and 6-gingerol. The capsaicinoid may be preferably at least one selected from capsaicin, dihydrocapsaicin, nordihydrocapsaicin, homodihydrocapsaicin, homocapsaicin, nonivamide and derivatives thereof and particularly preferably capsaicin.

The alkaloid may be preferably a piperidine derivative. The piperidine derivative may be preferably at least one selected from piperine and a derivative thereof and particularly preferably piperine.

### <TRPA1 Agonist>

A TRPA1 agonist is not particularly limited as long as it is a compound having an action to activate TRPA1 (transient receptor potential ankyrin 1). A TRPA1 agonist may be naturally or non-naturally occurring compound.

When a naturally occurring TRPA1 agonist is used, the TRPA1 agonist may be chemically synthesized or derived from a natural raw material, such as a plant and a microorganism, containing the TRPA1 agonist. The form of the TRPA1 agonist derived from a natural raw material is not particularly limited. The TRPA1 agonist may be present in the form of, e.g., a natural raw material, an extract from the natural raw material or a compound obtained from the natural raw material by purification or concentration.

The TRPA1 agonist may be preferably at least one selected from allyl isothiocyanate (AITC), cinnamaldehyde, diallyl disulfide, ASP7663 ((2E)-2-[7-fluoro-1,2-dihydro-1-(2-methylpropyl)-2-oxo-3H-indol-3-ylidene]acetic acid), oxylipin, diallyl sulfide, diallyl trisulfide, 1-menthol, allicin, acrolein, farnesylthiosalicylic acid, Δ₉-tetrahydrocannabinol, eugenol, sanshool, farnerthylthioacetic acid, ajoene, camphor, polygodial and methyl salicylate, and particularly preferably at least one selected from allyl isothiocyanate (AITC), cinnamaldehyde, diallyl disulfide, ASP7663 and oxylipin.

A preferable embodiment of oxylipin will be described below.

Oxylipin is a collective term of compounds produced by oxidatively metabolizing polyunsaturated fatty acids with an action of a plurality of enzymes including cyclooxygenase, lipoxygenase and an enzyme belonging to cytochrome P450 in vivo.

Oxylipin may consist only of a single compound or contain 2 or more compounds.

Oxylipin is a fatty acid typically having 12 to 26 carbon atoms, at least one carbon-carbon double bond and at least one oxygen-containing functional group selected from a hydroxy group, a hydroperoxide group, a keto group and an epoxy group. The carbon chain of the fatty acid may be a straight or branched chain or a straight or branched chain partially cyclized, and may preferably be a straight chain. The number of carbon atoms of the fatty acid may be preferably 16 to 22, more preferably 18 to 22, more preferably 18 to 20 and most preferably 18. The oxygen-containing functional group may be preferably at least one selected from a hydroxy group and a hydroperoxide group and more preferably a hydroxy group. The number of the oxygen-containing functional groups may be preferably 1 or 2 and more preferably 1. The number of the carbon-carbon double bonds may be preferably 1 to 6, more preferably 1 to 3, more preferably 2 or 3 and most preferably 3. The fatty acid may be present in the form of a salt.

Oxylipin can be, for example, at least one selected from (9Z,11E,15E)-13-hydroxy-9,11,15-octadecatrienoic acid, (7Z,13E,15Z)-12-hydroxy-7,13,15-octadecatrienoic acid, (9Z,13E,15Z)-12-hydroxy-9,13,15-octadecatrienoic acid, (9Z,12Z,14E)-16-hydroxy-9,12,14-octadecatrienoic acid and (10E,12Z,15Z)-9-hydroxy-10,12,15-octadecatrienoic acid. These oxylipins may be present in the form of a salt.

Oxylipin may be, for example, at least one selected from octadecatrienoic acid having a hydroxy group at the 12th position carbon having the following physicochemical properties:
(1) Appearance: light yellow transparent syrup-like solid
(2) HR MS [M+H]+: m/z 293.2116
(3) Molecular formula C₁₈H₃₀O₃
(4) Solubility: ethanol, methanol, acetonitrile, chloroform, ethyl acetate, DMSO (5) UV absorption spectrum: λmax nm 230 (in 80% CH₃CN)
(6) 1H NMR spectrum 1H-NMR (500 MHz, CHLOROFORM-D) δ 0.99 (t, J=7.7 Hz), 1.38-1.27 (m), 1.60-1.66 (m), 2.04 (m), 2.17-2.23 (m), 2.28-2.36 (m), 4.21 (q, J=6.3 Hz, 1H), 5.35-5.40 (m), 5.42-5.47 (m), 5.53-5.58 (m), 5.68 (dd, J=15.2, 6.6 Hz), 5.94 (t, J=11.2 Hz), 6.52 (dd, J=15.2, 11.2 Hz)
(7) 13C NMR spectrum 13C-NMR (126 MHz, CHLOROFORM-D) δ 178.5, 134.9, 134.7, 133.7, 127.2, 126.0, 124.5, 72.3, 35.4, 33.9, 29.5, 29.1, 29.0, 27.4, 24.7, 21.2, 14.3, and (9Z,11E,15E)-13-hydroxy-9,11,15-octadecatrienoic acid, (9Z,12Z,14E)-16-hydroxy-9,12,14-octadecatrienoic acid and (10E,12Z,15Z)-9-hydroxy-10,12,15-octadecatrienoic acid. The octadecatrienoic acid having a hydroxy group at the 12th position carbon may be, for example, (7Z,13E,15Z)-12-hydroxy-7,13,15-octadecatrienoic acid and/or (9Z,13E,15Z)-12-hydroxy-9,13,15-octadecatrienoic acid. These oxylipins may be present in the form of a salt.

Oxylipin may be derived from a natural raw material such as a plant, a microorganism and an animal, or chemically synthesized, and preferably derived from a natural raw material, particularly a plant. The plant may particularly preferably be lemongrass (scientific name: Cymbopogon citratus).

Oxylipin may be preferably in the form of a plant containing oxylipin, such as lemongrass, itself, or an extract from the plant, and particularly preferably a plant extract. When a plant itself is used, a part of plant such as leaf, stem and root, can be used.

Oxylipin may be a compound obtained from a natural raw material containing oxylipin by purification or concentration. Oxylipin obtained from a natural raw material by purification or concentration may be at least one predetermined compound of oxylipins contained in a natural raw material or all compounds of oxylipins contained in a plant raw material obtained by purification or concentration.

A plant extract containing oxylipin can be prepared by subjecting a plant raw material to an extraction operation using an extraction medium. The plant extract containing oxylipin may be preferably a hydrophobic extract of a plant. Examples of the extraction medium that can be used may include a solvent such as an organic solvent, water and hot water, and a supercritical fluid such as supercritical carbon dioxide. A solvent may be particularly preferred. The solvent may be particularly preferably a hydrophobic organic solvent such as ethanol, hexane and chloroform or a mixed solvent thereof. Of them, particularly ethanol and/or hexane may be preferred. When an extract is produced by extraction with a solvent, a plant raw material may be soaked in an appropriate amount of a solvent (for example, 0.5 to 100 fold on a weight basis relative to a plant raw material) and appropriately stirred or allowed to stand still to elute a component soluble in the solvent. Although it is not particularly limited, the extraction time can be 5 minutes to 24 hours, for example, 15 minutes to 15 hours. Although it is not particularly limited, the extraction temperature can be 0° C. to 125° C., for example 15° C. to 50° C. After completion of extraction, a solvent fraction containing a component soluble in the solvent and a solid fraction containing, e.g., cell wall, may be separated by a means for solid-liquid separation (for example, centrifugation, filtration (e.g., filtration by diatomaceous earth)) to obtain the solvent fraction as an extract. The extract obtained or a concentrate obtained by removing a solvent from the extract, may be directly used as an oxylipin-containing extract. Alternatively, the extract or concentrate, if necessary, may be further subjected to further purification means such as concentration, solvent fractionation, chromatography (e.g., column chromatography, gas chromatography, high performance liquid chromatography (HPLC), supercritical fluid chromatography) and/or recrystallization. A product obtained by treating oxylipin by purification or concentration may be used. The part of a plant raw material to be extracted, although it is not particularly limited, can be a part such as leaf, stem and root. A dried plant raw material may be preferred. For example, a lemongrass material to be used for preparation of a lemongrass extract containing oxylipin may be preferably dried leaf and/or stem of lemongrass. A plant raw material may be subjected to extraction in its original form. Alternatively, plant raw material may be cut into pieces having an appropriate size or form, crashed, pulverized, ground or squeezed, and then, subjected to extraction.

### <Composition for Enhancing Adrenomedullin Gene Expression>

One or more embodiments of the present invention relate to a composition for enhancing adrenomedullin gene expression, containing a TRPV1 agonist and a TRPA1 agonist as active ingredients.

Another or other embodiments of the present invention relate to a combination of a TRPV1 agonist and a TRPA1 agonist for use in enhancing adrenomedullin gene expression. The combination may be preferably a composition containing a TRPV1 agonist and a TRPA1 agonist or a kit containing a TRPV1 agonist and a TRPA1 agonist not mixed with each other.

Non Patent Literature 1 discloses that 6-shogaol has an action to promote release of adrenomedullin form the intestinal epithelial cells, and that the action is inhibited by a TRPA1 antagonist. However, Non Patent Literature 1 and other prior-art literatures do not suggest that an action to enhance adrenomedullin gene expression by a combination of a TRPV1 agonist and a TRPA1 agonist is a synergetic action that exceeds actions (additive action) expected from the actions by the TRPV1 agonist alone or a TRPA1 agonist alone.

The subject to which the composition or combination according to the embodiment to be applied may typically be a human. However, the subject is not limited to a human and may be another non-human animal, for example, a mammal except a human.

The subject to which the composition or combination according to the embodiment to be applied may be preferably a subject in need of enhancement of or being desirable to enhance adrenomedullin gene expression. The composition or combination according to the embodiment may also be effective for a healthy subject.

Adrenomedullin is a peptide having physiological activities such as vasodilation and angiogenesis. Blood flow can be increased by enhancing adrenomedullin gene expression. By virtue of an increase of blood flow by enhancement of adrenomedullin gene expression, it is possible to achieve an effect such as an antimicrobial action, anti-bowel inflammation, gastric mucosa protection and suppression of thrombogenesis. Because of this, the composition or combination according to the embodiment may be useful for achieving at least one effect selected from increasing blood flow, an antimicrobial action, an anti-bowel inflammation, gastric mucosa protection and suppression of thrombogenesis in a subject such as a human and a non-human mammal. The composition or combination according to the embodiment may be useful for treating or preventing a disease or symptom that is improved by enhancement of adrenomedullin gene expression, more specifically, at least one selected from a disease or symptom that is improved by an increase of blood flow, a disease or symptom caused by a microorganism, bowl inflammation, disease or symptom that is improved by gastric mucosa protection and disease or symptom that is improved by suppression of thrombogenesis, in a subject such as a human and a non-human mammal.

Enhancement of adrenomedullin gene expression can be confirmed by preparing cDNA from mRNA in cells taken from a subject to which the composition or combination according to the embodiment is fed or administered or cells cultured in the presence of the composition or combination according to the embodiment; performing a nucleic acid amplification reaction using the cDNA as a template and a primer set that can specifically amplify at least part of a cDNA nucleotide sequence (5′ untranslated region, coding region and/or 3′ untranslated region, preferably coding region) of adrenomedullin (more specifically, adrenomedullin precursor (preproadrenomedullin)); and detecting the amount of an amplified product by the reaction. Adrenomedullin is a peptide generated from a precursor (preproadrenomedullin). Because of this, “adrenomedullin gene expression” can also be referred to as “adrenomedullin precursor gene expression”.

Enhancement of adrenomedullin gene expression can be confirmed by detecting the amount of adrenomedullin peptide and the amount of an adrenomedullin precursor or the amount of peptides except adrenomedullin derived from an adrenomedullin precursor, preferably the amount of adrenomedullin peptide, in cells taken from a subject to which the composition or combination according to the embodiment is fed or administered or cells cultured in the presence of the composition or combination according to the embodiment. If the amount of adrenomedullin peptide, the amount of an adrenomedullin precursor or the amount of peptides except adrenomedullin derived from an adrenomedullin precursor, increases in cells, it can be evaluated that adrenomedullin gene expression is enhanced.

The amino acid sequence of a human-derived adrenomedullin precursor (preproadrenomedullin) is shown in SEQ ID NO: 3 and the cDNA nucleotide sequence of a human-derived adrenomedullin precursor (preproadrenomedullin) is shown in SEQ ID NO: 4. In SEQ ID NO: 4, a coding region corresponds to positions 179 to 736.

The content of a TRPV1 agonist and a TRPA1 agonist in the composition or combination according to the embodiment is not particularly limited. For example, the content of these agonists can be preferably 0.1 to 95 mass %, and more preferably 1 to 50 mass % based on the total amount of the composition or combination.

The blend ratio of a TRPV1 agonist and a TRPA1 agonist in the composition or combination according to the embodiment is not particularly limited. For example, in the composition or combination according to the embodiment, the content of a TRPA1 agonist per mole of a TRPV1 agonist may be preferably 0.1 to 100 moles, more preferably 0.1 to 50 moles, more preferably 0.1 to 20 moles, more preferably, 0.2 to 5 moles and particularly preferably 0.2 to 3 moles. If a TRPV1 agonist and a TRPA1 agonist each consist of a plurality of compounds, the number of moles of each of the TRPV1 agonist and TRPA1 agonist may be the total number of moles of plural compounds.

The composition or combination according to the embodiment preferably contains a TRPV1 agonist and a TRPA1 agonist such that the composition or combination can deliver the TRPV1 agonist in a concentration of 1 to 100 μM and the TRPA1 agonist in a concentration of 1 to 300 μM, and more preferably 1 to 100 μM, to cells or living tissue in which adrenomedullin gene expression is intended to be enhanced.

The composition or combination according to the embodiment may be a composition serving as a pharmaceutical, food and drink or a cosmetic. Examples of the food and drink may include food with functional claims, food for specified health use and supplements for nutrition supply.

The composition or combination according to the embodiment may be a composition or combination that is preferably orally or nasally fed or administered, and more preferably orally fed or administered.

The composition or combination according to the embodiment may be a composition or combination for use in enhancing adrenomedullin gene expression in cells in vitro by coexisting it with the cells. In this case, the composition or combination according to the embodiment may be used by adding it in a medium for culturing cells. The cells may be preferably epithelial cells, and more preferably small intestine epithelial cells.

The composition or combination according to the embodiment may be fed, administered or used continuously or as needed.

The state/form of the composition or combination according to the embodiment is not particularly limited. It may be presented in any state/form such as a liquid state, a fluid state, a gel state, a semi-solid form or a solid form.

The composition or combination according to the embodiment may further contain at least one additional component other than a TRPV1 agonist and a TRPA1 agonist. Although it is not particularly limited, the at least one additional component may be preferably a component that may be contained in the final form of a pharmaceutical, food and drink and a cosmetic and the like.

Examples of the additional component may include a sweetener, an acidulant, vitamins, minerals, a thickener, an emulsifier, an antioxidant and water. If necessary, e.g., a pigment, a flavor, a preservative, an antiseptic agent, a fungicide and another physiologically active substance may be added.

Examples of the sweetener may include monosaccharides such as glucose, fructose, sucrose, lactose, maltose, palatinose, trehalose and xylose, a disaccharide, an isomerized sugar (e.g., glucose syrup, high fructose corn syrup, sugar mixed isomerized sugar), a sugar alcohol (e.g., erythritol, xylitol, lactitol, palatinit, sorbitol, reduced starch syrup), honey and a high intensity sweetener (e.g., sucralose, acesulfame potassium, thaumatin, stevia, aspartame).

Examples of the acidulant may include citric acid, malic acid, gluconic acid, tartaric acid, lactic acid, phosphoric acid and salts thereof. Of these, one or two or more can be used.

Examples of the vitamins may include vitamin A, vitamin B1, vitamin B2, vitamin B6, vitamin E, niacin and inositol.

Examples of the minerals may include calcium, magnesium, zinc and iron.

Examples of the thickener may include carrageenan, gellan gum, xanthan gum, gum Arabic, tamarind gum, guar gum, locust bean gum, karaya gum, agar, gelatin, pectin, soybean polysaccharides and carboxymethyl cellulose (CMC).

Examples of the emulsifier may include a glycerin fatty acid ester, a sucrose fatty acid ester, a sorbitan fatty acid ester, lecithin, a plant sterol and saponin.

Examples of the antioxidant may include vitamin C, tocopherol (vitamin E) and enzyme-treated rutin.

The at least one additional component can be appropriately blended in an amount within the range conventionally employed by those skilled in the art in, e.g., food and drink compositions and pharmaceutical compositions.

The composition or combination according to the embodiment may contain only a TRPV1 agonist and a TRPA1 agonist as an active ingredient involved in enhancement of adrenomedullin gene expression. In this case, the composition or combination according to the embodiment may contain the at least one additional component as mentioned above not involved in enhancement of adrenomedullin gene expression.

A further another embodiment of the present invention relates to a medium composition for cell culture, comprising:
at least one medium component,
a TRPV1 agonist and
a TRPA1 agonist.

The medium composition may be used for culturing preferably epithelial cells and more preferably small intestine epithelial cells.

In the medium composition, the concentration of the TRPV1 agonist may preferably be 1 to 100 μM and the concentration of the TRPA1 agonist may preferably be 1 to 300 μM and more preferably 1 to 100 μM.

In the medium composition, the content of the TRPA1 agonist per mole of the TRPV1 agonist may be preferably 0.1 to 100 moles, more preferably 0.1 to 50 moles, more preferably 0.1 to 20 moles, more preferably 0.2 to 5 moles and particularly preferably 0.2 to 3 moles.

Examples of the at least one medium component may include at least one component conventionally used in mediums such as a nitrogen source, a carbon source and an inorganic salt.

### EXAMPLES

### Experiment 1: Enhancement of Adrenomedullin Gene Expression in Rat-Derived Small Intestinal Epithelial Cell Line by Combination of TRPV1 Agonist and TRPA1 Agonist

### <Test Compound>

The TRPV1 agonist used herein was 30 μM capsaicin (Product #034-11351, Wako Pure Chemical Industries Ltd.), 5 μM 6-shogaol (Product #192-16161, Wako Pure Chemical Industries Ltd.), 30 μM 6-gingerol (Product #076-05901, Wako Pure Chemical Industries Ltd.) or 30 μM piperine (Product #162-1724, FUJIFILM Wako Pure Chemical Corporation).

TRPA1 agonist used herein was 30 μM or 60 μM allyl isothiocyanate (AITC) (Product #016-01463, Wako Pure Chemical Industries Ltd.), 30 μM or 60 μM cinnamaldehyde (Product #031-03453, Wako Pure Chemical Industries Ltd.), 30 μM or 60 μM diallyl disulfide (Product #320-25071, Wako Pure Chemical Industries Ltd.) or 10 μM or 20 μM ASP7663 (Product #SML1467-5MG, Sigma-Aldrich).

### <Enhancement of Adrenomedullin Gene Expression>

A rat-derived small intestinal epithelial cell line (IEC-6) was suspended in DMEM (5% FBS, 4 μg/mL insulin) medium so as to obtain a density of 1×10⁵ cells/mL. The cell suspension was seeded (added to) in wells of a 24-well plate in an amount of 1 mL/well (1×10⁵ cells/well) and cultured at 37° C. for 24 hours in a 5% CO₂ condition.

Twenty-four hours after initiation of culture, the medium in each well was exchanged with DMEM medium containing a test compound in a predetermined concentration and then the cell line was cultured at 37° C. for further 6 hours in a 5% CO₂ condition.

A small intestinal epithelial cell line to be used as a control was cultured in the same manner as in the above except that DMEM medium not containing test compounds was used.

After completion of culture, cells were washed with phosphate buffer saline (PBS), a cell lysate was recovered by Buffer RLT supplied with RNeasy Mini Kit (QIAGEN).

Total RNA was prepared from the cell lysate by use of RNeasy Mini Kit (QIAGEN).

Real time PCR using total RNA was carried out to determine mRNA expression levels of adrenomedullin and internal standard β-actin. The real time PCR was carried out by One Step TB Green PrimeScript RT-PCR Kit II (Takara Bio Inc.) in accordance with the protocol supplied with the kit. The primer sequences are as shown in the following table.

TABLE 1

| | Forward primer (5′->3′) | Reverse primer (5′->3′) |
|---|---|---|
| adrenomedullin | GGCAGAACAACTCCAGCCTTTAC | ATCAGGGCGATGGAAACCAG |
| | (SEQ ID NO: 1) | (SEQ ID NO: 2) |
| β-actin | GGAGATTACTGCCCTGGCTCCTA | GACTCATCGTACTCCTGCTTGCTG |
| | (SEQ ID NO: 5) | (SEQ ID NO: 6) |

The mRNA expression level of adrenomedullin to that of β-actin was calculated to obtain a relative value. Furthermore, the mRNA expression levels of adrenomedullin calculated in individual conditions were expressed as relative values to the mRNA expression level (calculated) of adrenomedullin in the control condition.

In each condition, measurement was carried out three times and an average value and standard deviation were obtained. A posttest was carried out by multiple comparison according to one-way ANOVA and Tukey's HSD test.

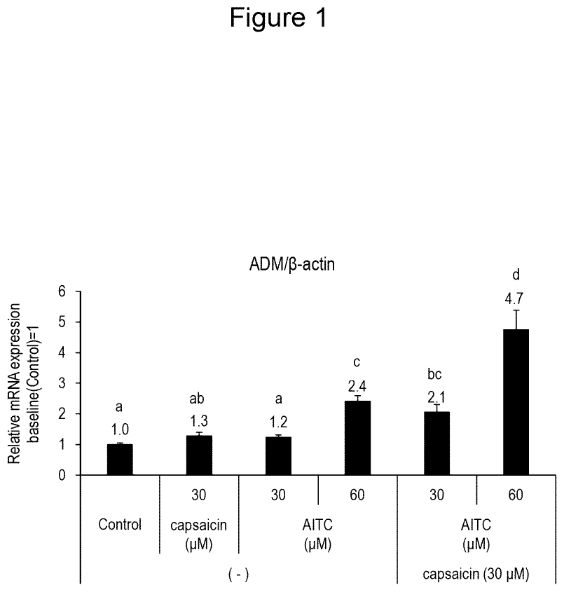

The results are shown in FIGS. 1 to 16. Conditions where a significant difference (p<0.05) is found were represented by different letters; whereas conditions where a significant difference is not found were represented by same letters.

FIG. 1 shows adrenomedullin mRNA expression levels in rat-derived small intestinal epithelial cell lines treated with 30 μM capsaicin alone, 30 μM or 60 μM AITC alone, or a combination of 30 μM capsaicin and 30 μM or 60 μM AITC. An increase of the adrenomedullin mRNA expression level of the cell line treated with the combination of 30 μM capsaicin and 30 μM or 60 μM AITC from that in the control condition exceeded a total of increases of adrenomedullin mRNA expression levels of the cell lines treated with individual test compounds alone from that in the control condition.

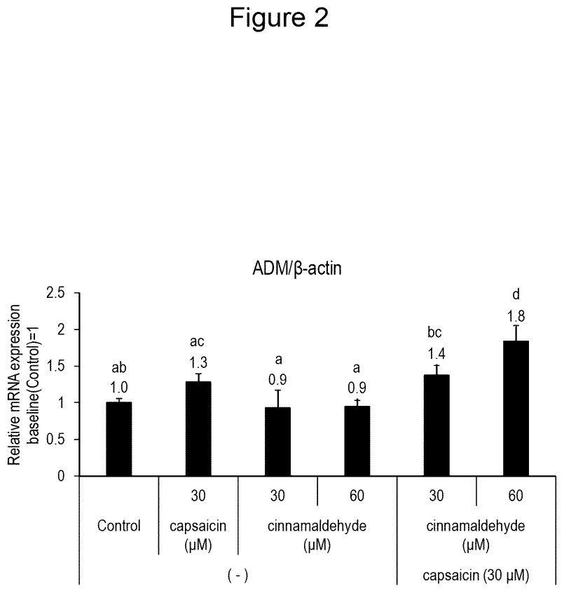

FIG. 2 shows adrenomedullin mRNA expression levels in rat-derived small intestinal epithelial cell lines treated with 30 μM capsaicin alone, 30 μM or 60 μM cinnamaldehyde alone, or a combination of 30 μM capsaicin and 30 μM or 60 μM cinnamaldehyde. An increase of the adrenomedullin mRNA expression level of the cell line treated with the combination of 30 μM capsaicin and 30 μM or 60 μM cinnamaldehyde from that in the control condition exceeded a total of increases of adrenomedullin mRNA expression levels of the cell lines treated with individual test compounds alone from that in the control condition.

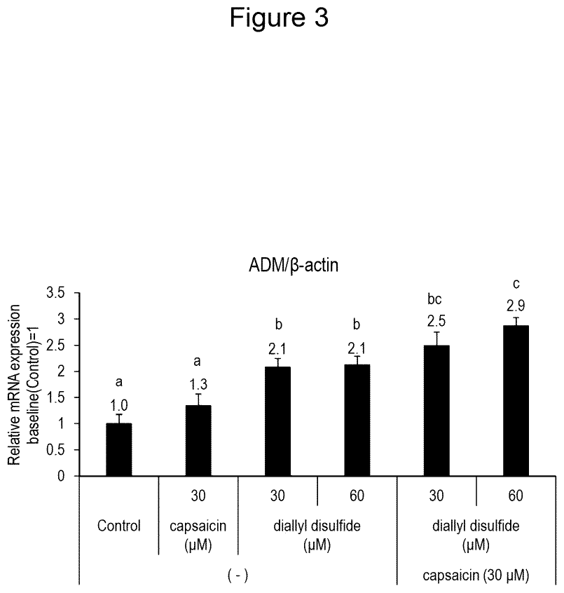

FIG. 3 shows adrenomedullin mRNA expression levels in rat-derived small intestinal epithelial cell lines treated with 30 μM capsaicin alone, 30 μM or 60 μM diallyl disulfide alone, or a combination of 30 μM capsaicin and 30 μM or 60 μM diallyl disulfide. An increase of the adrenomedullin mRNA expression level of the cell line treated with the combination of 30 μM capsaicin and 30 μM or 60 μM diallyl disulfide from that in the control condition exceeded a total of increases of adrenomedullin mRNA expression levels of the cell lines treated with individual test compounds alone from that in the control condition.

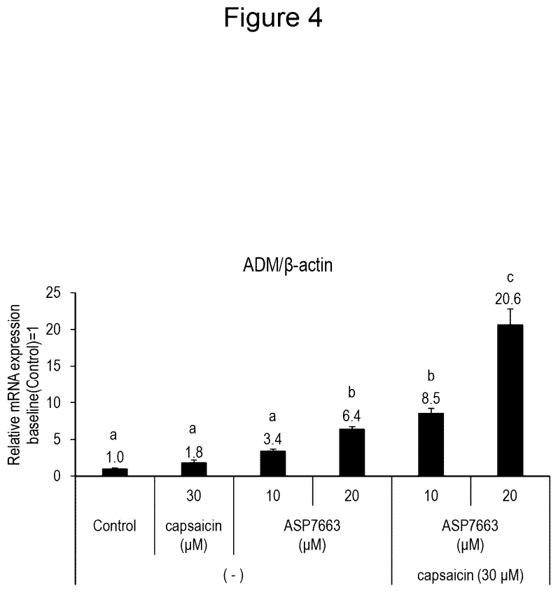

FIG. 4 shows adrenomedullin mRNA expression levels in rat-derived small intestinal epithelial cell lines treated with 30 μM capsaicin alone, 10 μM or 20 μM ASP7663 alone, or a combination of 30 μM capsaicin and 10 μM or 20 μM ASP7663. An increase of the adrenomedullin mRNA expression level of the cell line treated with the combination of 30 μM capsaicin and 10 μM or 20 μM ASP7663 from that in the control condition exceeded a total of increases of adrenomedullin mRNA expression levels of the cell lines treated with individual test compounds alone from that in the control condition.

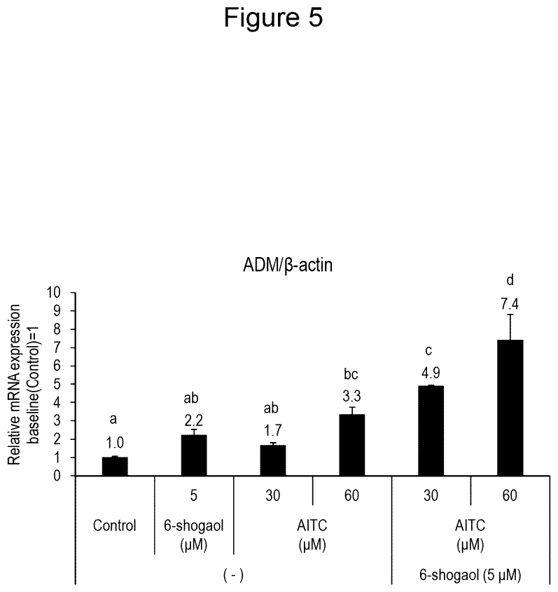

FIG. 5 shows adrenomedullin mRNA expression levels in rat-derived small intestinal epithelial cell lines treated with 5 μM 6-shogaol alone, 30 μM or 60 μM AITC alone, or a combination of 5 μM 6-shogaol and 30 μM or 60 μM AITC. An increase of the adrenomedullin mRNA expression level of the cell line treated with the combination of 5 μM 6-shogaol and 30 μM or 60 μM AITC from that in the control condition exceeded a total of increases of adrenomedullin mRNA expression levels of the cell lines treated with individual test compounds alone from that in the control condition.

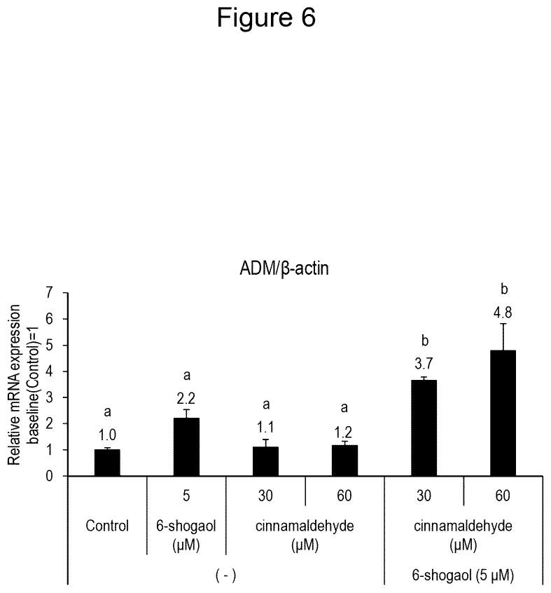

FIG. 6 shows adrenomedullin mRNA expression levels in rat-derived small intestinal epithelial cell lines treated with 5 μM 6-shogaol alone, 30 μM or 60 μM cinnamaldehyde alone, or a combination of 5 μM 6-shogaol and 30 μM or 60 μM cinnamaldehyde. An increase of the adrenomedullin mRNA expression level of the cell line treated with the combination of 5 μM 6-shogaol and 30 μM or 60 μM cinnamaldehyde from that in the control condition exceeded a total of increases of adrenomedullin mRNA expression levels of the cell lines treated with individual test compounds alone from that in the control condition.

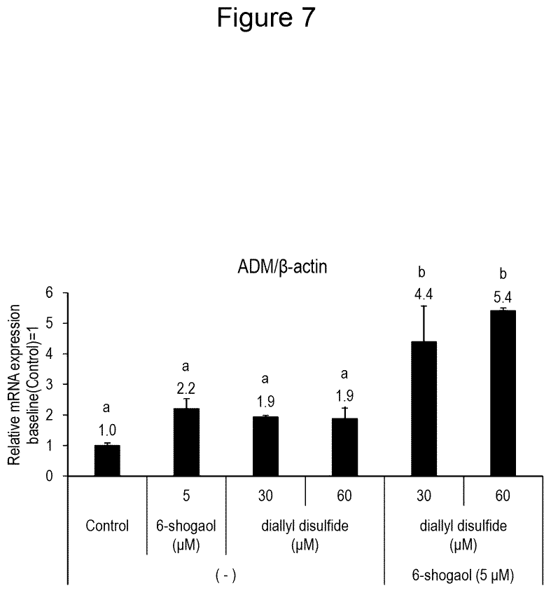

FIG. 7 shows adrenomedullin mRNA expression levels in rat-derived small intestinal epithelial cell lines treated with 5 μM 6-shogaol alone, 30 μM or 60 μM diallyl disulfide alone, or a combination of 5 μM 6-shogaol and 30 μM or 60 μM diallyl disulfide. An increase of the adrenomedullin mRNA expression level of the cell line treated with the combination of 5 μM 6-shogaol and 30 μM or 60 μM diallyl disulfide from that in the control condition exceeded a total of increases of adrenomedullin mRNA expression levels of the cell lines treated with individual test compounds alone from that in the control condition.

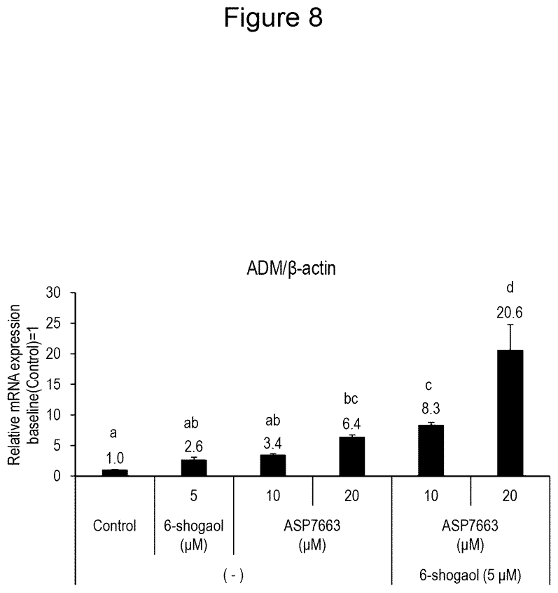

FIG. 8 shows adrenomedullin mRNA expression levels in rat-derived small intestinal epithelial cell lines treated with 5 μM 6-shogaol alone, 10 μM or 20 μM ASP7663 alone, or a combination of 5 μM 6-shogaol and 10 μM or 20 μM ASP7663. An increase of the adrenomedullin mRNA expression level of the cell line treated with the combination of 5 μM 6-shogaol and 10 μM or 20 μM ASP7663 from that in the control condition exceeded a total of increases of adrenomedullin mRNA expression levels of the cell lines treated with individual test compounds alone from that in the control condition.

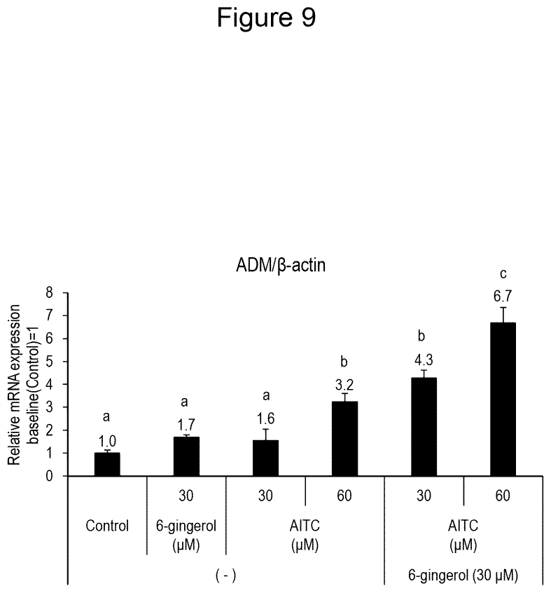

FIG. 9 shows adrenomedullin mRNA expression levels in rat-derived small intestinal epithelial cell lines treated with 30 μM 6-gingerol alone, 30 μM or 60 μM AITC alone, or a combination of 30 μM 6-gingerol and 30 μM or 60 μM AITC. An increase of the adrenomedullin mRNA expression level of the cell line treated with the combination of 30 μM 6-gingerol and 30 μM or 60 μM AITC from that in the control condition exceeded a total of increases of adrenomedullin mRNA expression levels of the cell lines treated with individual test compounds alone from that in the control condition.

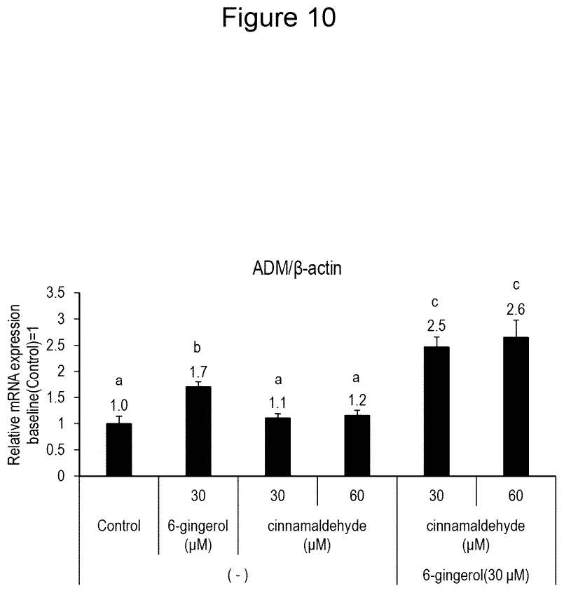

FIG. 10 shows adrenomedullin mRNA expression levels in rat-derived small intestinal epithelial cell lines treated with 30 μM 6-gingerol alone, 30 μM or 60 μM cinnamaldehyde alone, or a combination of 30 μM 6-gingerol and 30 μM or 60 μM cinnamaldehyde. An increase of the adrenomedullin mRNA expression level of the cell line treated with the combination of 30 μM 6-gingerol and 30 μM or 60 μM cinnamaldehyde from that in the control condition exceeded a total of increases of adrenomedullin mRNA expression levels of the cell lines treated with individual test compounds alone from that in the control condition.

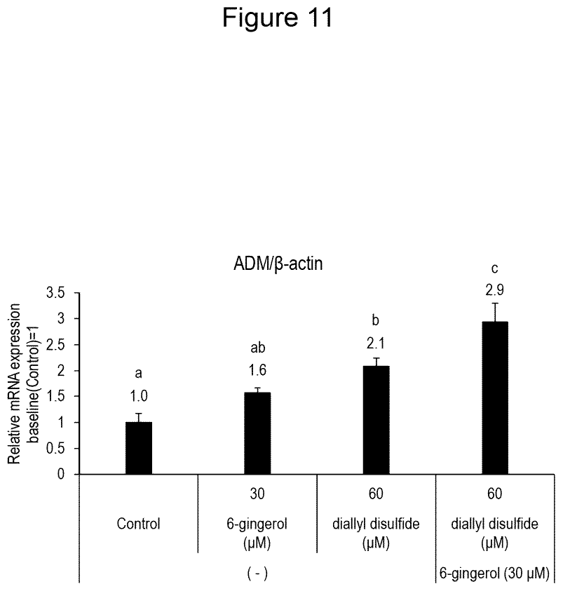

FIG. 11 shows adrenomedullin mRNA expression levels in rat-derived small intestinal epithelial cell lines treated with 30 μM 6-gingerol alone, 60 μM diallyl disulfide alone, or a combination of 30 μM 6-gingerol and 60 μM diallyl disulfide. An increase of the adrenomedullin mRNA expression level of the cell line treated with the combination of 30 μM 6-gingerol and 60 μM diallyl disulfide from that in the control condition exceeded a total of increases of adrenomedullin mRNA expression levels of the cell lines treated with individual test compounds alone from that in the control condition.

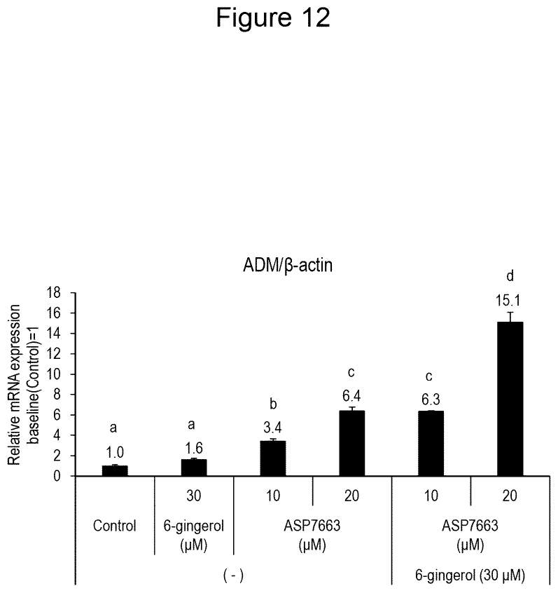

FIG. 12 shows adrenomedullin mRNA expression levels in rat-derived small intestinal epithelial cell lines treated with 30 μM 6-gingerol alone, 10 μM or 20 μM ASP7663 alone, or a combination of 30 μM 6-gingerol and 10 μM or 20 μM ASP7663. An increase of the adrenomedullin mRNA expression level of the cell line treated with the combination of 30 μM 6-gingerol and 10 μM or 20 μM ASP7663 from that in the control condition exceeded a total of increases of adrenomedullin mRNA expression levels of the cell lines treated with individual test compounds alone from that in the control condition.

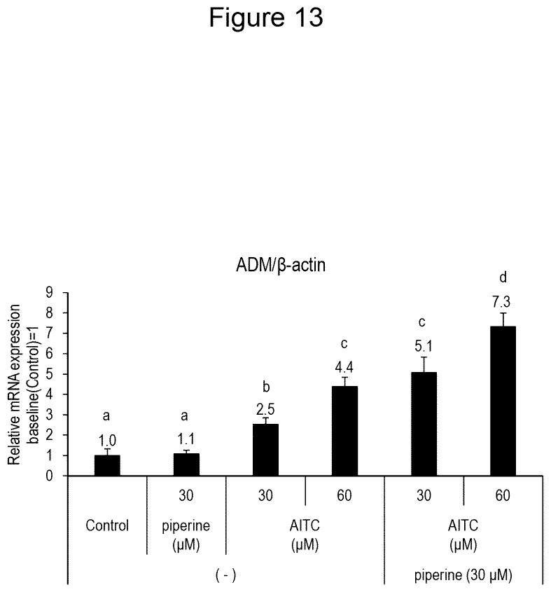

FIG. 13 shows adrenomedullin mRNA expression levels in rat-derived small intestinal epithelial cell lines treated with 30 μM piperine alone, 30 μM or 60 μM AITC alone, or a combination of 30 μM piperine and 30 μM or 60 μM AITC. An increase of the adrenomedullin mRNA expression level of the cell line treated with the combination of 30 μM piperine and 30 μM or 60 μM AITC from that in the control condition exceeded a total of increases of adrenomedullin mRNA expression levels of the cell lines treated with individual test compounds alone from that in the control condition.

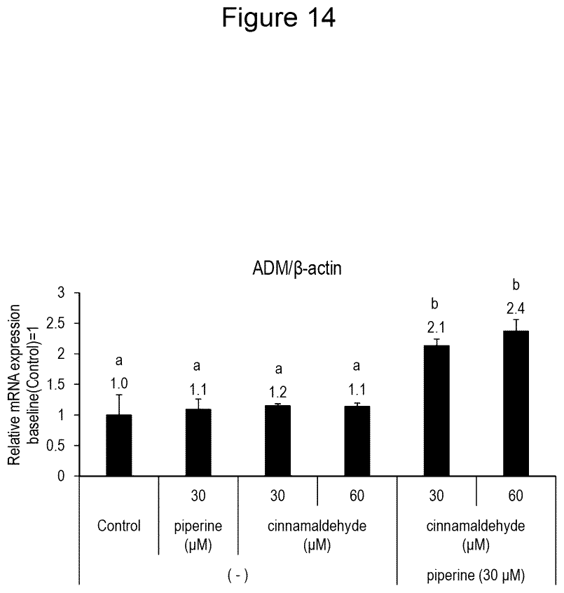

FIG. 14 shows adrenomedullin mRNA expression levels in rat-derived small intestinal epithelial cell lines treated with 30 μM piperine alone, 30 μM or 60 μM cinnamaldehyde alone, or a combination of 30 μM piperine and 30 μM or 60 μM cinnamaldehyde. An increase of the adrenomedullin mRNA expression level of the cell line treated with the combination of 30 μM piperine and 30 μM or 60 μM cinnamaldehyde from that in the control condition exceeded a total of increases of adrenomedullin mRNA expression levels of the cell lines treated with individual test compounds alone from that in the control condition.

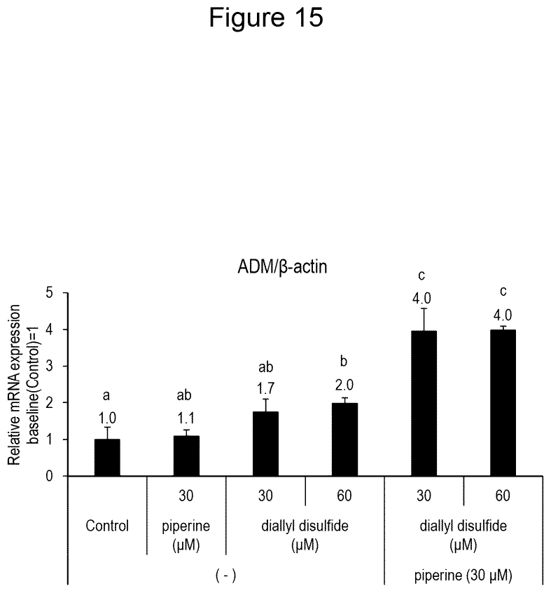

FIG. 15 shows adrenomedullin mRNA expression levels in rat-derived small intestinal epithelial cell lines treated with 30 μM piperine alone, 30 μM or 60 μM diallyl disulfide alone, or a combination of 30 μM piperine and 30 μM or 60 μM diallyl disulfide. An increase of the adrenomedullin mRNA expression level of the cell line treated with the combination of 30 μM piperine and 30 μM or 60 μM diallyl disulfide from that in the control condition exceeded a total of increases of adrenomedullin mRNA expression levels of the cell lines treated with individual test compounds alone from that in the control condition.

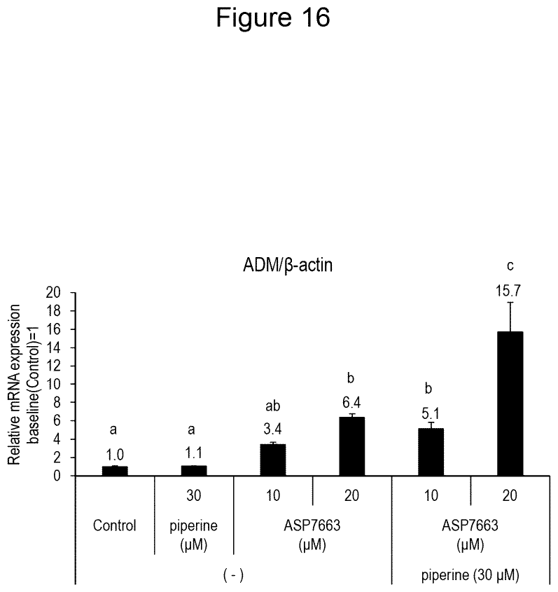

FIG. 16 shows adrenomedullin mRNA expression levels in rat-derived small intestinal epithelial cell lines treated with 30 μM piperine alone, 10 μM or 20 μM ASP7663 alone, or a combination of 30 μM piperine and 10 μM or 20 μM ASP7663. An increase of the adrenomedullin mRNA expression level of the cell line treated with the combination of 30 μM piperine and 10 μM or 20 μM ASP7663 from that in the control condition exceeded a total of increases of adrenomedullin mRNA expression levels of the cell lines treated with individual test compounds alone from that in the control condition.

The above results demonstrate that the action to increase adrenomedullin mRNA expression level in rat-derived small intestinal epithelial cell line exerted by a combination of a TRPV1 agonist and a TRPA1 agonist is a synergetic action, regardless of types of compounds (agonists) selected, exceeding a total (additive action) of the actions to increase adrenomedullin mRNA expression level exerted by the TRPV1 agonist alone and TRPA1 agonist alone.

### Experiment 2: Analysis of Oxylipin Contained in Lemongrass Extract

(1) Extraction of Oxylipin from Lemongrass

To 2 L of chloroform in a conical flask, about 100 g of lemongrass dry powder was added. Extraction was carried out at room temperature for 12 hours or more while stirring by a stirrer. The resultant supernatant was collected by suction filtration and concentrated to dryness by a rotary evaporator.

### (2) Partial Purification of Oxylipin

The resulting concentrated/dried solid substance (2 to 5 g) was purified by a medium-pressure liquid chromatographic (LC) apparatus in the following conditions. The eluate was collected in units of 15 ml/fraction.

### LC Apparatus

Silica gel injection column: W830
Medium-pressure LC apparatus: EPCLC-W-Prep 2XY (Yamazen Co., Ltd.)
Purification column: Si-40D (silica gel column)

### Mobile Phase Conditions

Mobile phase: hexane/ethyl acetate
0 minute (50%/50%), 16 minutes (50%/50%), 32 minutes (10%/90%), 48 minutes (10%/90%)
Flow rate: 40 ml/min

Fractions obtained during the retention time of 28 minutes to 40 minutes were subjected to thin-layer chromatographic (TLC) analysis in the following conditions. The fractions where a spot exhibiting an Rf value of 0.5 was detected were collected and combined as a partially purified oxylipin fraction.

TLC conditions are as follows.
TLC plate: Silica gel TLC plate, product name: Glass Plate Silica Gel 60 F₂₅₄, product No: 105715 (Merck Millipore)
Developing solvent: chloroform:methanol=9:1
Coloring reagent: phosphomolybdic acid coloring reagent (ethanol solution containing 10% of sodium phosphomolybdate n-hydrate)

### (3) Purification of Oxylipin

The partially purified oxylipin fraction was subjected to preparative TLC in the following conditions. An absorption band (detected) at a UV wavelength of 254 nm and having an Rf value of 0.5 was scraped/collected and soaked in an extraction solvent (chloroform:methanol=8:2) having a volume ratio of 5 times or more. The solution was stirred by a stirrer for 30 minutes or more to extract a component. The extract was filtered with filter paper. The filtrate was concentrated to dryness by a rotary evaporator. The resulting concentrated/dried solid substance was regarded as purified oxylipin.

Preparative TLC conditions are as follows.
TLC plate: Preparative TLC plate, Product name: PLC Glass Plate Silica Gel 60 F254, 1 mm, Model Number: 113895 (Merck Millipore)
Developing solvent: chloroform:methanol=9:1

### (4) Isolation of Various Types of Oxylipins

Purified oxylipin was subjected to recycling preparative HPLC in the following conditions. Compounds detected at three discrete sites were individually collected. The individual fractions collected were designated as R1, R2 and R3 in the order from the shortest retention time.

### Conditions of the First Recycling Preparative HPLC

Recycling preparative (HPLC) apparatus: LC-9110 (Japan Analytical Industry Co., Ltd.)
Preparative column: Diol column, product name: Inertsil (registered trademark) Diol, particle size 5 μm, inner diameter 14 mm, length 250 mm, model number: 5020-79054 (GL Sciences)
Mobile phase: hexane:ethanol=9:1
Flow rate: 9.999 ml/min
Detection wavelength: 230 nm,
Recycle count: 8 or more

Subsequently, R1, R2 and R3 were separately subjected to recycling preparative HPLC in the following conditions. From each of the fractions detected, one or two compounds were individually collected.

### Conditions for the Second Recycling Preparative HPLC:

Recycling preparative device: LC-9110 (Japan Analytical Industry Co., Ltd.)
Preparative column: Diol column, product name: Inertsil (registered trademark) Diol, particle size 5 μm, inner diameter 14 mm, length 250 mm, model number: 5020-79054 (GL Sciences)
Mobile phase: chloroform:methanol=98:2, 99:1 or 97:3
Flow rate: 9.999 ml/min
Detection wavelength: 230 nm
Recycle count: 8 or more

### (5) Structural Determination

Two compounds separated from R1 were designated as R1-1 and R1-2. Two compounds separated from R2 were designated as R2-1 and R2-2. A single compound separated from R3 was designated as R3. Of them, compounds except R1-1 (present in an ultratrace amount) were subjected to structural analysis.

Compound R1-2 isolated was dried by an evaporator and then analyzed by NMR and HPLC-Orbitrap™MS system (Thermo Fisher Scientific). As a result, R1-2 was identified as a compound ((9Z,11E,15E)-13-hydroxy-9,11,15-octadecatrienoic acid) having a planar configuration represented by the following formula.

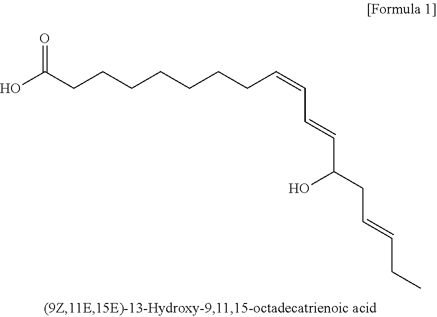 [SMILES: CC/C=C/CC(O)/C=C/C=C\CCCCCCCC(=O)O]

### Compound R1-2 Isolated has the Following Physicochemical Properties:

(1) Appearance: light yellow transparent syrup-like solid
(2) HR MS [M+H]+: m/z 293.2118
(3) Molecular formula C₁₈H₃₀O₃
(4) Solubility: ethanol, methanol, acetonitrile, chloroform
(5) UV absorption spectrum: λmax nm 230 (in 80% CH₃CN)
(6) 1H NMR spectrum (500 MHz, CHLOROFORM-D) δ 0.96 (t, J=7.7 Hz), 1.37 (q, J=6.5 Hz), 1.60-1.66 (m), 2.04-2.09 (m), 2.15-2.19 (m), 2.28-2.38 (m), 4.23 (q, J=6.3 Hz), 5.35 (q, J=9.5 Hz), 5.41-5.46 (m), 5.54-5.59 (m), 5.68 (dd, J=15.2, 6.6 Hz), 5.97 (t, J=11.2 Hz), 6.52 (dd, J=15.8, 11.7 Hz)
(7) 13C NMR spectrum (126 MHz, CHLOROFORM-D) δ 14.3, 20.8, 24.7, 27.6, 28.9, 29.4, 33.8, 35.3, 72.2, 123.8, 126.0, 127.9, 133.0, 134.9, 135.4, 178.1

Compound R2-1 isolated was dried by an evaporator and thereafter analyzed by NMR and HPLC-Orbitrap™MS system (Thermo Fisher Scientific). As a result, R2-1 was estimated as either one or a mixture of (7Z,13E,15Z)-12-hydroxy-7,13,15-octadecatrienoic acid and (9Z,13E,15Z)-12-hydroxy-9,13,15-octadecatrienoic acid having a planar configuration represented by the following formula:

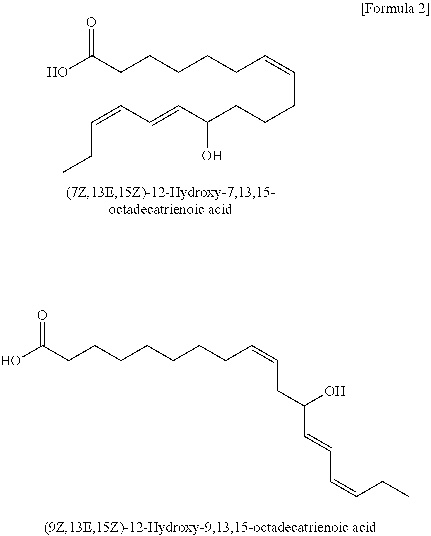 [SMILES: CC/C=C\C=C\C(O)CCC/C=C\CCCCCC(=O)O ; CC/C=C\C=C\C(O)C/C=C\CCCCCCCC(=O)O]

### Compound R2-1 Isolated has the Following Physicochemical Properties:

(1) Appearance: light yellow transparent syrup-like solid
(2) HR MS [M+H]+: m/z 293.2116
(3) Molecular formula C₁₈H₃₀O₃
(4) Solubility: ethanol, methanol, acetonitrile, chloroform, ethyl acetate, DMSO
(5) UV absorption spectrum: λmax nm 230 (in 80% CH₃CN)
(6) 1H NMR spectrum 1H-NMR (500 MHz, CHLOROFORM-D) δ 0.99 (t, J=7.7 Hz), 1.38-1.27 (m), 1.60-1.66 (m), 2.04 (m), 2.17-2.23 (m), 2.28-2.36 (m), 4.21 (q, J=6.3 Hz, 1H), 5.35-5.40 (m), 5.42-5.47 (m), 5.53-5.58 (m), 5.68 (dd, J=15.2, 6.6 Hz), 5.94 (t, J=11.2 Hz), 6.52 (dd, J=15.2, 11.2 Hz)
(7) 13C NMR spectrum 13C-NMR (126 MHz, CHLOROFORM-D) δ 178.5, 134.9, 134.7, 133.7, 127.2, 126.0, 124.5, 72.3, 35.4, 33.9, 29.5, 29.1, 29.0, 27.4, 24.7, 21.2, 14.3

Compound R2-2 isolated was dried by an evaporator and then analyzed by NMR and HPLC-Orbitrap™MS system (Thermo Fisher Scientific). As a result, R2-2 was identified as a compound ((9Z,12Z,14E)-16-hydroxy-9,12,14-octadecatrienoic acid) having a planar configuration represented by the following formula:

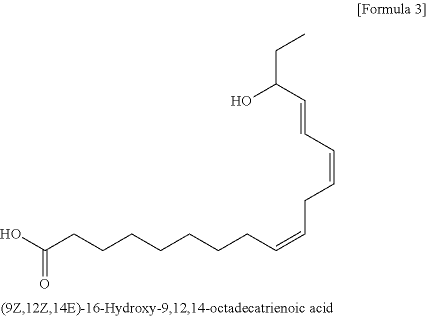 [SMILES: CCC(O)/C=C/C=C\C/C=C\CCCCCCCC(=O)O]

### Compound R2-2 Isolated has the Following Physicochemical Properties:

(1) Appearance: light yellow transparent syrup-like solid
(2) HR MS [M+H]+: m/z 293.2116
(3) Molecular formula C₁₈H₃₀O₃
(4) Solubility: ethanol, methanol, acetonitrile, ethyl acetate, chloroform
(5) UV absorption spectrum: λmax nm 230 (in 80% CH₃CN)
(6) 1H NMR spectrum (500 MHz, CHLOROFORM-D) δ 6.52 (dd, J=14.9, 10.9 Hz), 5.99 (t, J=10.9 Hz), 5.68 (dd, J=15.2, 6.6 Hz), 5.40 (q, J=8.0 Hz), 5.39-5.31 (m), 4.12 (q, J=6.5 Hz), 2.92 (t, J=7.2 Hz), 2.34 (t, J=7.4 Hz), 2.05 (q, J=6.9 Hz), 1.66-1.59 (m), 1.60-1.55 (m), 1.25-1.39 (m), 0.93 (t, J=7.4 Hz, 3H)
(7) 13C NMR spectrum (126 MHz, CHLOROFORM-D) δ 178.4, 135.9, 130.8, 127.9, 127.3, 125.8, 77.4, 77.1, 76.9, 74.3, 33.8, 30.3, 29.5, 29.1, 29.0, 27.2, 26.2, 24.7, 9.8

Compound R3 isolated was dried by an evaporator and then analyzed by NMR and HPLC-Orbitrap™MS system (Thermo Fisher Scientific). As a result, R3 was identified as a compound ((10E,12Z,15Z)-9-hydroxy-10,12,15-octadecatrienoic acid) having a planar configuration represented by the following formula:

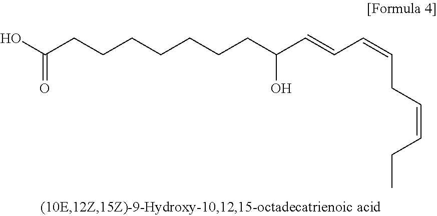 [SMILES: CC/C=C\C/C=C\C=C\C(O)CCCCCCCC(=O)O]

### Compound R3 Isolated has the Following Physicochemical Properties:

(1) Appearance: light yellow transparent syrup-like solid
(2) HR MS [M+H]+: m/z 293.2117
(3) Molecular formula C₁₈H₃₀O₃
(4) Solubility: ethanol, methanol, acetonitrile, ethyl acetate, chloroform
(5) UV absorption spectrum: λmax nm 230 (in 80% CH₃CN)
(6) 1H NMR spectrum (500 MHz, CHLOROFORM-D) δ 6.50 (dd, J=14.9, 10.9 Hz), 5.98 (t, J=10.9 Hz), 5.68 (dd, J=14.9, 6.9 Hz), 5.46-5.38 (m), 5.31 (q, J=8.8 Hz), 4.16 (q, J=6.5 Hz), 2.92 (t, J=7.4 Hz), 2.34 (t, J=7.7 Hz), 2.10-2.04 (m), 1.64-1.61 (m), 1.57-1.50 (m), 0.97 (t, J=7.4 Hz)
(7) 13C NMR spectrum (126 MHz, CHLOROFORM-D) δ 178.7, 136.3, 132.5, 130.9, 127.9, 126.6, 125.6, 73.0, 37.3, 33.9, 29.4, 29.2, 29.0, 26.1, 25.4, 24.7, 20.7, 14.3

### Experiment 3: Measurement of TRP Channel Agonist Activity

### (1) Measuring Method

### Construction of Vector

DNA fragments containing nucleotide sequences encoding amino acid sequences of human TRPA1 (Accession No. NM_007332.3) (hereinafter referred to also as hTRPA1), human TRPV1 (Accession No. NM_080704.4) (hereinafter referred to also as hTRPV1) and human TRPM8 (Accession No. NM_024080.5) (hereinafter referred to also as hTRPM8) were inserted into pcDNA5/TO vectors (Invitrogen) to construct an hTRPA1 expression vector, an hTRPV1 expression vector and an hTRPM8 expression vector, respectively.

Acquisition of hTRPA1, hTRPV1 and hTRPM8 Expressing Cells

T-REx™_293 cells (Invitrogen) were cultured in Dulbecco's modified Eagle medium (DMEM, Nacalai Tesque Inc.) containing 10% fetal bovine serum (FBS, Cytiva) up to 60 to 70% confluent growth in the wells of a 6 well plate. T-REx™-293 cells were transfected with an hTRPA1 expression vector, an hTRPV1 expression vector or an hTRPM8 expression vector by use of Lipofectamine LTX (Invitrogen) in accordance with a manual.

### Acquisition of Stably Expressing Cell Line

To Dulbecco's modified Eagle medium (DMEM, Nacalai Tesque Inc.) containing 10% fetal bovine serum (FBS, Cytiva), hygromycin B and blasticidin S were added so as to have a concentration of 400 μ/ml and 5 μg/ml, respectively. In this manner, a drug selection medium was prepared. Twenty-four hours after completion of transfection, the medium of the above cells was exchanged with the drug selection medium, and thereafter, the cells were cultured for 7 days while exchanging the medium with a fresh medium every two days. Living cells were subcultured and regarded as hTRPA1 stably expressing cell line, hTRPV1 stably expressing cell line and hTRPM8 stably expressing cell line, respectively.

### Preparation of Intracellular Ca²⁺ Imaging Method

The day before assay, cells of the individual expressing cell lines were removed from the medium with a trypsin/EDTA solution and seeded in the wells of a 96-well black-well plate with a clear bottom so as to obtain a cell density of 5×10⁴ cells to 6×10⁴ cells/well. At this time, tetracycline was added to the medium so as to obtain a concentration of 1 μg/ml to induce expression of hTRPA1, hTRPV1 or hTRPM8. Culture was carried out in an incubator at 37° C. for 24 hours to 48 hours and then cells were washed with PBS. After PBS was removed from the wells, 50 μl of a calcium fluorescent indicator, Calbryte 520 AM (AAT, Bioquest), which was prepared by dissolving CaCl₂ dihydrate, HEPES, BSA in an assay buffer HBSS (−) (solution prepared by adding so as to obtain a final concentration of 1 mM, 20 mM and 0.1%, respectively), so as to obtain a concentration of 4 μM, was added, to the wells and incubated at 33° C. for 45 minutes. Thereafter, the cells were again washed with the assay buffer and then 100 μl was allowed to remain in the wells to prepare cells for assay. A solution of a test substance for evaluating TRP channel agonist activity and having a concentration twice as large as the concentration for measurement was prepared within the wells of the 96-well plate and used within 30 minutes after preparation. The cells to be subjected to assay were incubated at 31° C. and a test substance solution (100 μl) was added to the cells for assay by use of Flaxstation3 (Molecular Devices) and fluorescence intensity (excitation wavelength: 490 nm, fluorescence wavelength: 525 nm) was measured every 2 seconds up to 120 seconds. Thirty seconds after initiation of measurement, the (test substance) solution was added to the cells for assay such that a final concentration of the test substance becomes a concentration for measurement.

TRP channel activation ability of a test substance was calculated as a relative value based on the activation ability of 10 μM ionomycin to each of TRP channels, in accordance with the following calculation formula.

Activation rate (%)=(test substance maximum fluorescence intensity−baseline)/(ionomycin maximum fluorescence intensity−baseline)×100
Test substance maximum fluorescence intensity: maximum fluorescence intensity value after addition of a test substance
Baseline: Average fluorescence intensity value 20 seconds after initiation of measurement
Ionomycin maximum fluorescence intensity: maximum fluorescence intensity value after addition of ionomycin

When TRPA1 activation ability was measured, a TRPA1 specific antagonist, A-967079, was added simultaneously with a test substance so as to obtain a final concentration of 1 μM, in order to confirm that a test substance specifically activates TRPA1. Also, whether the activity is suppressed or not was determined.

### (2)-1: Evaluation 1

In Experiment 2 (4), the TRPA1 activation abilities of R2 fraction of oxylipin obtained in the first preparative HPLC, 10 μM ionomycin (IONO), 30 μM allyl isothiocyanate (AITC) and 100 μM cinnamaldehyde (CA) were measured. TRPA1 activation ability of the case where 0.02% of dimethyl sulfoxide (DMSO) was added in place of a test substance was measured as a negative control and the activation rate thereof based on (the activity of) 10 μM ionomycin was obtained.

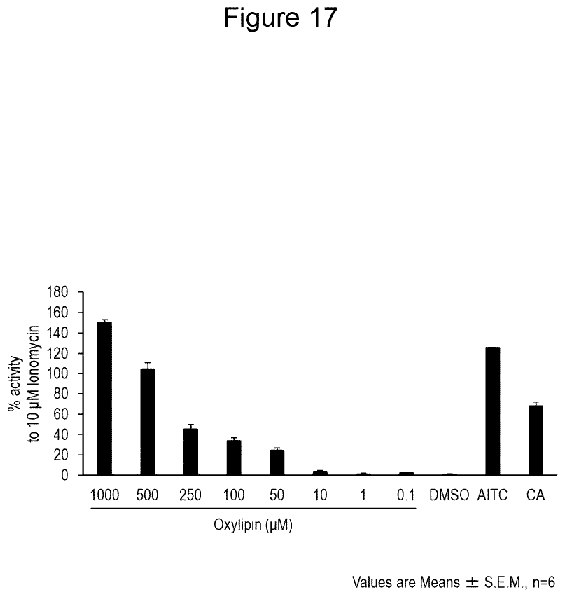

With respect to R2 fraction of oxylipin (molecular weight was regarded as 294 g/mol), TRPA1 activation ability was measured in the concentration range of 1 μM to 1000 μM. The results are shown in FIG. 17. The results of FIG. 17 demonstrate that oxylipin has a TRPA1 agonist activity.

### (2)-2: Evaluation 2

TRPA1 activation abilities of 100 μM and 500 μM purified oxylipins (molecular weight was regarded as 294 g/mol) prepared in Experiment 2 (3) and 30 μM allyl isothiocyanate (AITC) were measured in the presence or absence of 1 μM A-967079 and activation rates thereof based on (the activity of) 10 μM ionomycin were obtained. In the tests carried out in the absence of A-967079, dimethyl sulfoxide (DMSO) was added in place of A-967079. TRPA1 activation ability of the case where DMSO was added in place of a test substance was measured as a negative control.

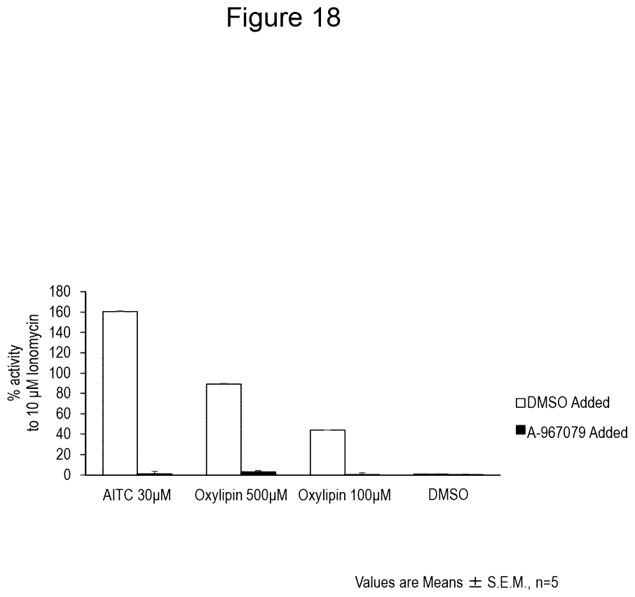

The results are shown in FIG. 18. 100 μM and 500 μM purified oxylipins did not show TRPA1 activation ability in the presence of an antagonist A-967079, and showed TRPA1 activation ability in the absence of A-967079, similarly to 30 μM allyl isothiocyanate (AITC) known as a TRPA1 agonist.

### (2)-3: Evaluation 3

In Experiment 2 (4), the TRPV1 activation abilities of R2 fraction (molecular weight was regarded as 294 g/mol) of 100 μM and 500 μM oxylipin obtained in the first preparative HPLC and 1 μM capsaicin were measured and activation rates based on (the activity of) 10 μM ionomycin were obtained. TRPV1 activation ability of the case where dimethyl sulfoxide (DMSO) was added in place of a test substance was measured as a negative control, and activation rate thereof based on (the activity of) 10 μM ionomycin was obtained.

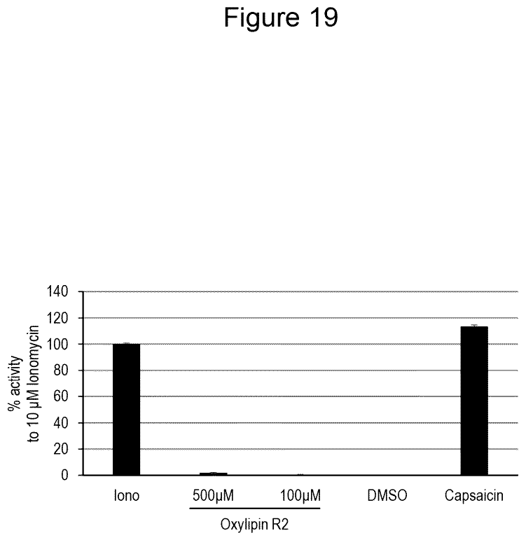

The results are shown in FIG. 19. It was confirmed that R2 fraction of oxylipin has substantially no TRPV1 agonist activity.

### (2)-4: Evaluation 4

In Experiment 2 (4), the TRPM8 activation abilities of R2 fraction (molecular weight was regarded as 294 g/mol) of 100 μM and 500 μM oxylipin obtained in the first preparative HPLC and 5 μM icilin were measured and activation rates thereof based on (the activity of) 10 μM ionomycin were obtained. TRPM8 activation ability of the case where dimethyl sulfoxide (DMSO) was added in place of a test substance was measured as a negative control and activation rate thereof based on (the activity of) 10 μM ionomycin was obtained.

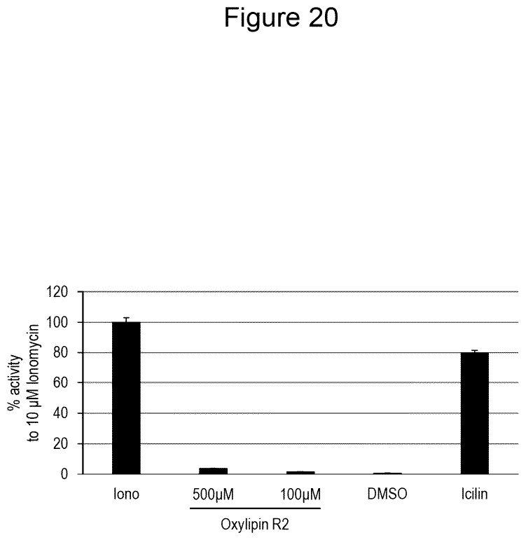

The results are shown in FIG. 20. It was confirmed that R2 fraction of oxylipin has substantially no TRPM8 agonist activity.

### (2)-5: Evaluation 5

In Experiment 2 (4), the TRPA1 activation abilities of 200 μM and 500 μM compounds R1-2, R2-1, R2-2 and R3 and 30 μM allyl isothiocyanate (AITC) obtained in the second preparative HPLC were measured in the presence or absence of 1 μM A-967079 and the activation rates thereof based on (the activity of) 10 μM ionomycin were obtained. In the test carried out in the absence of A-967079, dimethyl sulfoxide (DMSO) was added in place of A-967079. TRPA1 activation ability of the case where DMSO was added in place of a test substance was measured as a negative control.

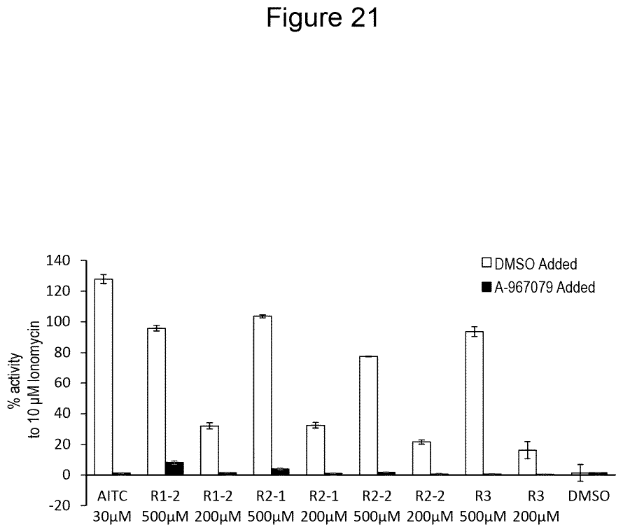

The results are shown in FIG. 21. Oxylipin compounds R1-2, R2-1, R2-2 and R3 did not show TRPA1 activation ability in the presence of an antagonist A-967079 and showed TRPA1 activation ability in the absence of A-967079 in a concentration dependent manner, similarly to 30 μM allyl isothiocyanate (AITC) known as a TRPA1 agonist.

### Experiment 4: Synergistic Action of Oxylipin and 6-Shogaol Used in Combination

### <Test Compound>

As a TRPV1 agonist, 5 μM 6-shogaol (Product #192-16161, Wako Pure Chemical Industries Ltd.) was used.

As a TRPA1 agonist, 50 μM, 83.3 μM and 250 μM purified oxylipins (molecular weight was regarded as 294 g/mol) prepared in Experiment 2 (3) or 20 μM ASP7663 (Product #SML1467-5MG, Sigma-Aldrich) were used.

In the same procedure as in Experiment 1, adrenomedullin mRNA expression level of rat-derived small intestinal epithelial cell lines in the presence of either one or both of a TRPV1 agonist having a predetermined concentration and a TRPA1 agonist having a predetermined concentration was calculated as a relative value to mRNA expression level of β-actin. Furthermore, mRNA expression levels of adrenomedullin calculated in individual conditions were expressed as a relative value to the mRNA expression level of adrenomedullin calculated in the control condition.

Measurement in each of the conditions was repeated three times (n=3) and an average value and standard deviation were obtained. A posttest was carried out by multiple comparison according to one-way ANOVA and Tukey's HSD test.

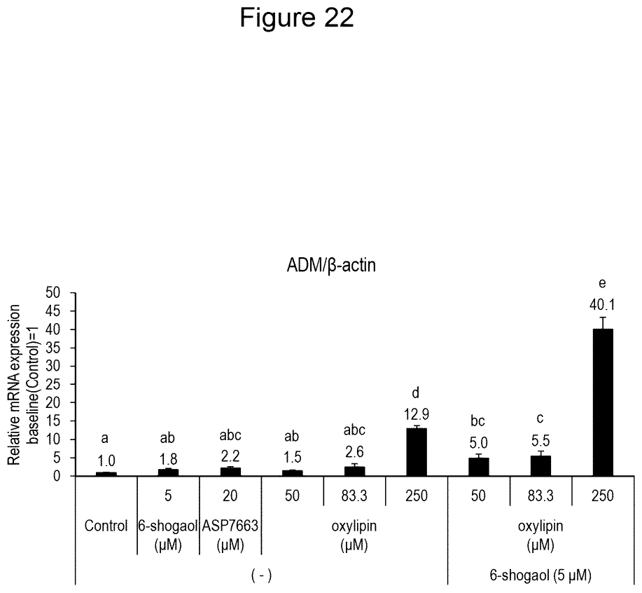

The results are shown in FIG. 22. Conditions where significant difference of p<0.05 is observed are represented by different letters and the conditions where no significant difference is observed are represented by same letters.

FIG. 22 shows adrenomedullin mRNA expression levels in rat-derived small intestinal epithelial cell lines treated with 5 μM 6-shogaol alone, 20 μM ASP7663 alone, 50 μM, 83.3 μM and 250 μM purified oxylipins alone, or combinations of 5 μM 6-shogaol and 50 μM, 83.3 μM or 250 μM purified oxylipin. An increase of the adrenomedullin mRNA expression level of the cell line treated with the combination of 5 μM 6-shogaol and 50 μM, 83.3 μM or 250 μM purified oxylipins from that in the control condition exceeded a total of increases of adrenomedullin mRNA expression levels of the cell lines treated with individual test compounds alone from that in the control condition.

All publications, patents and patent applications cited herein are incorporated in their entireties by reference.

1. A method for enhancing adrenomedullin gene expression, comprising administering a TRPV1 agonist and a TRPA1 agonist to a subject in need of enhancement of or being desirable to enhance adrenomedullin gene expression.

2. The method according to claim 1, wherein the TRPV1 agonist is at least one selected from capsaicin, 6-shogaol, 6-gingerol and piperine.

3. The method according to claim 1, wherein the TRPA1 agonist is at least one selected from allyl isothiocyanate, cinnamaldehyde, diallyl disulfide, ASP7663 and oxylipin.

4. The method according to claim 1, wherein the subject is a human or a non-human mammal.

5. A method for enhancing adrenomedullin gene expression in cells in vivo or in vitro, comprising providing a TRPV1 agonist and a TRPA1 agonist to cells in vivo or in vitro.

6. The method according to claim 5, wherein the cells are epithelial cells.


SEQUENCE LISTING (6 sequences)

SEQ ID NO: 1 - DNA, 23 bp, synthetic construct
  misc_feature 1..23: note = primer
  source 1..23: mol_type = other DNA
ggcagaacaactccagcctttac

SEQ ID NO: 2 - DNA, 20 bp, synthetic construct
  misc_feature 1..20: note = primer
  source 1..20: mol_type = other DNA
atcagggcgatggaaaccag

SEQ ID NO: 3 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 4 - DNA, 1492 bp, Homo sapiens
  source 1..1492: mol_type = unassigned DNA
actcagtggtttcttggtgacactggatagaacagctcaagccttgccacttcgggcttctcactgcagctgggcttggacttcggagttttgccattgccagtgggacgtctgagactttctccttcaagtacttggcagatcactctcttagcagggtctgcgcttcgcagccgggatgaagctggtttccgtcgccctgatgtacctgggttcgctcgccttcctaggcgctgacaccgctcggttggatgtcgcgtcggagtttcgaaagaagtggaataagtgggctctgagtcgtgggaagagggaactgcggatgtccagcagctaccccaccgggctcgctgacgtgaaggccgggcctgcccagacccttattcggccccaggacatgaagggtgcctctcgaagccccgaagacagcagtccggatgccgcccgcatccgagtcaagcgctaccgccagagcatgaacaacttccagggcctccggagctttggctgccgcttcgggacgtgcacggtgcagaagctggcacaccagatctaccagttcacagataaggacaaggacaacgtcgcccccaggagcaagatcagcccccagggctacggccgccggcgccggcgctccctgcccgaggccggcccgggtcggactctggtgtcttctaagccacaagcacacggggctccagcccccccgagtggaagtgctccccactttctttaggatttaggcgcccatggtacaaggaatagtcgcgcaagcatcccgctggtgcctcccgggacgaaggacttcccgagcggtgtggggaccgggctctgacagccctgcggagaccctgagtccgggaggcaccgtccggcggcgagctctggctttgcaagggcccctccttctgggggcttcgcttccttagccttgctcaggtgcaagtgccccagggggcggggtgcagaagaatccgagtgtttgccaggcttaaggagaggagaaactgagaaatgaatgctgagacccccggagcaggggtctgagccacagccgtgctcgcccacaaactgatttctcacggcgtgtcaccccaccagggcgcaagcctcactattacttgaactttccaaaacctaaagaggaaaagtgcaatgcgtgttgtacatacagaggtaactatcaatatttaagtttgttgctgtcaagattttttttgtaacttcaaatatagagatatttttgtacgttatatattgtattaagggcattttaaaagcaattatattgtcctcccctattttaagacgtgaatgtctcagcgaggtgtaaagttgttcgccgcgtggaatgtgagtgtgtttgtgtgcatgaaagagaaagactgattacctcctgtgtggaagaaggaaacaccgagtctctgtataatctatttacataaaatgggtgatatgcgaacagcaaaccaataaactgtctcaatgctga

SEQ ID NO: 5 - DNA, 23 bp, synthetic construct
  misc_feature 1..23: note = primer
  source 1..23: mol_type = other DNA
ggagattactgccctggctccta

SEQ ID NO: 6 - DNA, 24 bp, synthetic construct
  misc_feature 1..24: note = primer
  source 1..24: mol_type = other DNA
gactcatcgtactcctgcttgctg